(19) United States
(12) Patent Application Publication
(10) Pub. No.: US 2026/0369355 A1
(43) Pub. Date: Nov. 19, 2026

(54) GENE SEQUENCE SCREENING METHOD AND SYSTEM OF TUMOR MARKERS

(71) Applicant: THE SECOND AFFILIATED HOSPITAL OF GUANGZHOU MEDICAL UNIVERSITY, Guangzhou (CN)

(72) Inventors: Sicong HUANG, Guangzhou (CN); Yongjie SHI, Guangzhou (CN); Yimei CHEN, Guangzhou (CN); Hongyun JIA, Guangzhou (CN); Qiwen XIAO, Guangzhou (CN); Cong DENG, Guangzhou (CN); Qiang ZHOU, Guangzhou (CN); Tianxing JI, Guangzhou (CN); Pei YU, Guangzhou (CN); Jiale KANG, Guangzhou (CN)

(73) Assignee: THE SECOND AFFILIATED HOSPITAL OF GUANGZHOU MEDICAL UNIVERSITY, Guangzhou (CN)

(21) Appl. No.: 19/724,969

(22) Filed: Jun. 30, 2026

Related U.S. Application Data
(63) Continuation of application No. PCT/CN2025/106387, filed on Jul. 1, 2025.

(30) Foreign Application Priority Data
Nov. 20, 2024 (CN) 2024116581889

(51) Int. Cl. G16B 30/00

(52) U.S. Cl. CPC G16B 30/00

(57) ABSTRACT

The present disclosure discloses a gene sequence screening method and system for tumor markers. The method comprises: acquiring a gene sequence to be analyzed and anoriginal gene sequence of each tumor marker, and segmenting the gene sequence to be analyzed and original gene sequence acquired into gene data blocks based on different lengths; calculating a partitioning evaluation value according to similarity and difference between data blocks at the same length, and adaptively determining an optimal block length by comparing the differences between evaluation values at adjacent lengths; and segmenting the gene sequence to be analyzed, and performing hash matching between the segmenting results and segmenting results corresponding to tumor markers in the database to achieve the precise screening of marker genes in patients with digestive tract tumors.

### CROSS-REFERENCE TO RELATED APPLICATIONS

The application claims priority to Chinese patent application No. 2024116581889, filed on Nov. 20, 2024, the entire contents of which are incorporated herein by reference.

### TECHNICAL FIELD

The present disclosure relates to the technical field of bioinformatics, in particular to a gene sequence screening method and system for tumor markers.

### BACKGROUND

During tumor detection, human tissues and body fluids can be extracted to acquire gene products from human metabolism. The acquired gene products are compared with gene data of tumor markers in established gene libraries. Analysis of the concentrations of different markers in the patient's genes can provide data support for relevant medical staff.

Labeling and matching can be performed through hash values when comparing the gene sequences of patients to be tested with the gene sequences of tumor markers, thereby achieving rapid gene alignment. However, during hash value retrieval, due to the diversity of gene sequences, minor variations may occur during gene transcription, such as deletions or errors that alter a small number of bases. These slight changes can lead to substantial differences in hash values, resulting in mismatches and compromising the accuracy of gene alignment. Therefore, block-based matching is adopted for gene sequences. Nevertheless, data blocks that are either too large or too small can affect alignment outcomes and reduce the accuracy of gene sequence screening.

### SUMMARY

To address the technical problem that improper blocking of gene sequences in the prior art leads to inaccurate gene alignment and unreliable gene sequence screening results, the present disclosure aims to provide a gene sequence screening method and system for tumor markers. The detailed technical solutions are set forth as follows:

In a first aspect, the present disclosure provides a gene sequence screening method for tumor markers, the method comprising the following steps:
acquiring a gene sequence to be analyzed from a patient with suspected digestive tract tumors and each original gene sequence of each tumor marker stored in a database, and iteratively partitioning the original gene sequences with different preset lengths to obtain gene data blocks of each original gene sequence at each length;
obtaining a partitioning evaluation value of each original gene sequence at each length according to the matching degree of data similarity and difference between gene data blocks of each original gene sequence and gene data blocks of other original gene sequences for the same tumor marker at the same length;
obtaining an optimal length according to the change in differences between the partitioning evaluation values of all original gene sequences at each length and partitioning evaluation values of all original gene sequences at an adjacent length; and
partitioning the gene sequence to be analyzed by using the optimal length, and performing hash matching between the partitioning results and partitioning results corresponding to tumor markers in the database to obtain the gene sequence screening results of tumor markers in the patient with suspected digestive tract tumors.

Preferably, the step of obtaining a partitioning evaluation value of each original gene sequence at each length according to the matching degree of data similarity and difference between gene data blocks of each original gene sequence and gene data blocks of other original gene sequences for the same tumor marker at the same length specifically comprises:
for any type of tumor marker and any length;
determining a global similarity feature value of each gene data block in each original gene sequence according to the similarity between each gene data block in each original gene sequence and each gene data block in every other original gene sequences;
screening each gene data block in the original gene sequence based on the global similarity feature value to determine a difference data block;
obtaining a partitioning evaluation value of each original gene sequence at any length according to the maximum similarity between each difference data block in each original gene sequence and correspondingly matching data blocks in other original gene sequences.

Preferably, the step of determining the global similarity feature value of each gene data block in each original gene sequence according to the similarity between each gene data block in each original gene sequence and each gene data block in every other original gene sequence specifically comprises:
taking any gene data block in any original gene sequence as a target data block, and taking the gene data block at the same corresponding position as the target data block in every other original gene sequence except any of the original gene sequences as a reference data block of the target data block; wherein, the target data block and the reference data block are gene data corresponding to the same type of tumor marker;
determining a similarity feature factor between the target data block and each reference data block based on the dynamic time warping (DTW) distance between the target data block and each reference data block; and
determining the global similarity feature value of the target data block based on the number of corresponding reference data blocks when the similarity feature factor is greater than a preset similarity threshold, wherein the global similarity feature value is a normalized value.

Preferably, the step of screening each gene data block in the original gene sequence based on the global similarity feature value to determine a difference data block specifically comprises:
determining the target data block as the difference data block when the global similarity feature value of the target data block is less than a preset difference threshold.

Preferably, the step of obtaining the partitioning evaluation value of each original gene sequence at any length according to the maximum similarity between each difference data block in each original gene sequence and the correspondingly matching data block in other original gene sequences specifically comprises:
determining the partitioning characteristic factor of each difference data block based on the negative correlation coefficient of the maximum value of the similarity feature factor between each difference data block and the corresponding reference data block in the original gene sequence for any original gene sequence at any length; and
determining the partitioning evaluation value of any original gene sequence at any length by integrating the partitioning characteristic factors of all difference data blocks in the original gene sequence.

Preferably, the step of obtaining the optimal length according to the change in differences between the partitioning evaluation values of all original gene sequences at each length and the partitioning evaluation values of all original gene sequences at an adjacent length specifically comprises:
obtaining a preference level of each length according to the change in differences of partitioning evaluation values of all original gene sequences between each length and an adjacent length in ascending order of length, wherein a minimum length corresponding to a preference level greater than a preset preference threshold is used as the optimal length.

Preferably, the step of obtaining a preference level of each length according to the change in differences of partitioning evaluation values of all original gene sequences between each length and an adjacent length specifically comprises:
calculating a mean value of partitioning evaluation values of all original gene sequences at each length to obtain a partitioning effect characterization value of each length; determining a partitioning effect slope value of each length based on a change rate of a partitioning effect characterization value between each length and a previous adjacent length in ascending order of length; and
for any length, determining a feature evaluation coefficient of the length based on the negative correlation coefficient of partitioning effect slope value difference between the length and the previous adjacent length, and obtaining a preference level of the length according to the feature evaluation coefficient and the partitioning effect characterization value of the length, wherein the feature evaluation coefficient and the partitioning effect characterization value are both positively correlated with the preference level.

Preferably, the step of performing hash matching between the gene sequence to be analyzed and the partitioning results corresponding to tumor markers in the database according to the partitioning results to obtain the gene sequence screening result of tumor markers of patients with digestive tract disorders specifically comprises:
for any tumor marker, taking the difference data block corresponding to the minimum value of the global similarity feature value at the optimal length as a seed data block, and performing hash matching based on the seed data block and each data block in the partitioning result of the gene sequence to be analyzed to obtain the matching result between the gene sequence to be analyzed and any of the tumor markers; and
acquiring the concentration proportion of each tumor marker in the gene sequence to be analyzed based on the matching result of each tumor marker, and determining the gene sequence screening result of tumor markers according to the descending order of concentration.

Preferably, the matching result between the gene sequence to be analyzed and any of the tumor markers is specifically as follows:
when the proportion of data blocks matching the seed data block in the partitioning result of the gene sequence to be analyzed is greater than the preset proportion threshold, matching the original gene sequence where the seed data block is located with the gene sequence to be analyzed to obtain the matching result of tumor markers.

In a second aspect, the present disclosure provides a gene sequence screening system for tumor markers, comprising:
a data extraction module, configured to acquire a gene sequence to be analyzed from a patient with suspected digestive tract tumors and each original gene sequence of each tumor marker stored in a database, and iteratively partition original gene sequences by using different preset lengths to obtain gene data blocks of each original gene sequence at each length;
a segmentation processing module, configured to obtain a partitioning evaluation value of each original gene sequence at each length according to the matching of data similarity and difference between gene data blocks of each original gene sequence and other original gene sequences for the same type of tumor markers at the same length;
a feature evaluation module, configured to obtain the optimal length according to the change in differences between partitioning evaluation values of all original gene sequences at each length and partitioning evaluation values of all original gene sequences at an adjacent length; and
a gene screening module, configured to partition the gene sequence to be analyzed using the optimal length, and perform hash matching between the partitioning result of the gene sequence to be analyzed and the partitioning result corresponding to the tumor marker in the database, so as to obtain the gene sequence screening results of tumor markers for a patient with suspected digestive tract tumors.

The embodiments of the present disclosure at least have the following beneficial effects:

In the present disclosure, the to-be-analyzed gene sequences to be analyzed from patients for gene alignment and the original gene sequences stored in the database are acquired, and iterative blocking and partitioning is performed on the gene information in the database using different length values, thereby laying a data foundation for subsequent analysis of partitioning feature corresponding to different lengths. Then, based on the data performance of gene information of the same type of tumor markers in the database under blocking results of different lengths, the gene similarity and difference are analyzed to quantitatively evaluate the blocking effect of each original gene sequence at each length, namely the partitioning evaluation value. Herein, similarity reflects the common data expression among different gene sequences, while difference reflects the unique data expression thereof. Further, based on the partitioning evaluation results of all original gene sequences at different lengths and combined with the evaluation results of adjacent lengths, the variation differences of partitioning under different lengths are analyzed to select the optimal option and realize the adaptive determination of the optimal length. Finally, the gene sequence to be analyzed is partitioned with the optimal length and matched with the partitioning results of the database under the same optimal length. This method can achieve favorable blocking outcomes, improve the accuracy and reference value of gene alignment, and thus obtain superior gene screening results.

### BRIEF DESCRIPTION OF THE DRAWINGS

To clearly illustrate the technical solutions and advantages in the embodiments of the present disclosure or in the prior art, the accompanying drawings required for describing the embodiments or the prior art are briefly introduced below. Obviously, the drawings described below merely show some embodiments of the present disclosure, and those skilled in the art can derive other drawings from these drawings without creative efforts.

FIG. 1 is a step flowchart of a gene sequence screening method for tumor markers provided in the present disclosure;

FIG. 2 is a step flowchart of an acquisition method of a partitioning evaluation value of an original gene sequence provided in the present disclosure;

FIG. 3 is a step flowchart of an acquisition method of an optimal length provided in the present disclosure;

FIG. 4 is a local distribution coordinate graph of a partitioning evaluation value of an original gene sequence provided in the present disclosure;

FIG. 5 is a system block graph of a gene sequence screening system for tumor markers provided in the present disclosure; and

FIG. 6 is a structural schematic diagram of a computer device provided in the present disclosure.

### DETAILED DESCRIPTION OF THE EMBODIMENTS

To further elaborate on the technical solutions and effects adopted by the present disclosure to achieve the intended objectives, the specific implementations, structures, features and effects of the gene sequence screening method and system of the tumor marker proposed herein are described in detail below in conjunction with the accompanying drawings and preferred embodiments. In the following description, references to “one embodiment” or “another embodiment” do not necessarily refer to the same embodiment. In addition, the specific features, structures or characteristics of one or more embodiments may be combined in any appropriate manner.

Unless otherwise defined, all technical and scientific terms used herein shall have the same meanings as commonly understood by those skilled in the art to which the present disclosure pertains.

The specific solutions of the gene sequence screening method and system for tumor markers provided by the present disclosure are described in detail below with reference to the accompanying drawings.

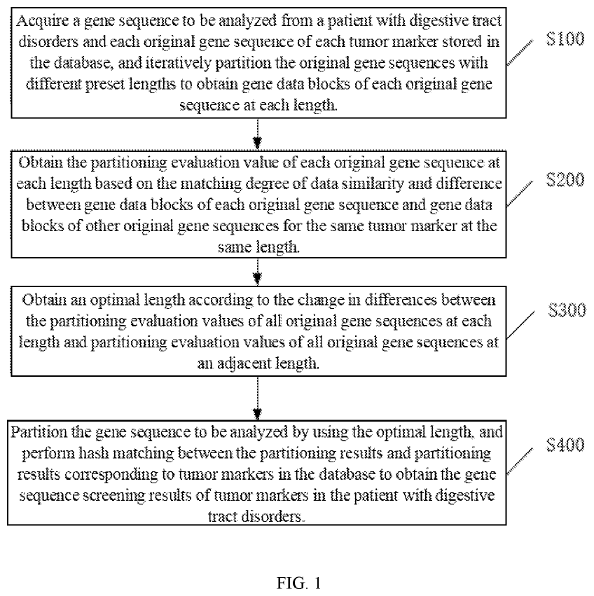

Please refer to FIG. 1, which illustrates a step flowchart of a gene sequence screening method for tumor markers according to an embodiment of the present disclosure. The method comprises the following steps:

Step S100, a gene sequence to be analyzed from a patient with suspected digestive tract tumors and each original gene sequence of each tumor marker stored in a database are acquired, and the original gene sequences are iteratively partitioned by adopting different preset lengths to obtain gene data blocks of each original gene sequence at each length.

When the human body suffers from tumor diseases, it secretes corresponding proteins or experiences elevated concentrations of specific substances, which are referred to as tumor markers. The synthesis of the tumor marker is generally realized through protein expression guided by gene sequences. By comparing and screening the genes corresponding to the tumor markers, the expression of relevant gene sequences can be preliminarily determined. During gene comparison and screening, gene sequences can be partitioned into blocks for alignment. However, excessively large blocks fail to deliver the intended effect, and mutations or errors will still impact hash values. If the blocks are too small, identical genetic information is likely to be detected at multiple positions in the gene sequence, which also undermines the accuracy of gene alignment. A longer data block tends to increase the probability of errors in matching the patient's gene sequence, while a shorter data block results in lower uniqueness within the entire gene library.

Accordingly, this embodiment first acquires the protein expression data of patients to be subjected to gene alignment. Meanwhile, gene information of each type of tumor marker in the gene database is processed into blocks of different lengths, which lays a data foundation for the subsequent adaptive analysis of blocking performance with varying block sizes.

Specifically, first, the gene sequences to be analyzed from patients with digestive tract disorders are acquired, and multiple distinct gene expressions corresponding to each type of tumor marker are retrieved from the database and defined as original gene sequences. Namely, various gene expression forms of a single tumor marker may be stored in the database, so multiple original gene sequences are correspondingly associated with the same tumor marker.

In this embodiment, the gene sequences in the gene library are iteratively partitioned into blocks at different lengths. The iterative range of the length is (0,1000), and the step size between two adjacent length iterations is set to 5. It can be understood that all length values are integers and increased sequentially according to the iteration step size. In other embodiments, the minimum length may also be set to the length of the base, the smallest genetic unit of the gene sequences in the gene library. Starting from this initial length, iterative increment is performed to partition the original gene sequences into blocks of varying lengths.

It should be noted that for each selected length, each original gene sequence of each type of tumor marker is partitioned into blocks to obtain genetic data blocks. This further provides a data basis for the subsequent analysis of the blocking results of different original gene sequences of the same tumor marker under a fixed block length.

Specifically, the genetic information of patients with digestive tract disorders is extracted as follows: approximately 10 grams of stool specimens collected via colonoscopy and before surgery are obtained using stool specimen containers, and a fecal occult blood test is immediately performed. Reagent preparation: 90 mL of isopropanol is added to buffer GFA, 135 mL and 160 mL of absolute ethanol are respectively added to a deproteinization solution RD and a rinse solution PWD. The preparation time is recorded and labels are affixed accordingly. 0.2 g of fecal sample was transferred into a 2 mL centrifuge tube A (200 μL of liquid sample was pipetted). 500 μL of Buffer SA, 100 μL of Buffer SC and 0.25 g of grinding beads were added sequentially. The mixture is thoroughly mixed by vortexing and then lysed in a metal bath at 70 °C for 15 min. Upon completion of lysis, the centrifuge tube is centrifuged at 12000 rpm for 1 minute, and the supernatant is transferred into a new 2 mL centrifuge tube B. 200 μL of Buffer SH was added into centrifuge tube B, followed by vortexing for thorough mixing. The mixture was incubated at 4 °C for 10 min and then centrifuged at 12000 rpm for 2 min. The supernatant was transferred to a 2 mL centrifuge tube C. Subsequently, 500 μL of Buffer GFR and 10 μL of magnetic bead suspension G were added, and the mixture was vortexed for 2 minutes to achieve complete mixing. The centrifuge tube C was placed on a magnetic stand and allowed to stand for 30 s. After the magnetic beads were fully adsorbed onto the inner wall, all liquid was carefully aspirated. The centrifuge tube C was removed, and 700 μL of deproteinization solution RD was added. The mixture was vortexed for 3 min. The tube was then placed on a magnetic stand and left to stand for 30 s. After the magnetic beads were fully adsorbed to the inner wall, all liquid was carefully aspirated. The centrifuge tube C was taken down, and 700 μL of deproteinization solution PWD was added. The mixture was vortexed for 3 min. The tube was placed on a magnetic stand and kept still for 30 s. After the magnetic beads were fully adsorbed onto the inner wall, all liquid was carefully aspirated. The centrifuge tube C was not removed at this step and was air-dried at room temperature for 5 min. The centrifuge tube C was taken down, and 80 μL of eluent TB was added. The mixture was vortexed for 1 min and then incubated in a metal bath at 56 °C for 5 min. The centrifuge tube C was placed on a magnetic stand and allowed to stand for 2 min. After the magnetic beads were completely adsorbed onto the inner wall, the DNA solution was carefully aspirated into a new centrifuge tube D.

Step S200, the partitioning evaluation value of each original gene sequence at each length is obtained according to the matching of data similarity and the differences between gene data blocks of each original gene sequence and other original gene sequences for the same type of tumor markers with identical lengths.

When the gene sequence screening of the tumor markers is performed, the gene information of the tumor markers in the known gene library is compared with the acquired gene information of patients with digestive tract disorders. Combined with the results of base information comparison using hash values, the matching status of gene information from patients to be tested can be determined. After the original gene sequences are segmented according to different lengths, the dynamic time warping algorithm can be adopted to perform data matching analysis on gene data blocks at each corresponding position, so as to quantify the evaluation results of segmentation effects under different lengths.

Meanwhile, considering that the patient gene information to be matched needs to be compared one by one with all gene information of the same type of tumor markers during gene information alignment. However, the original gene sequences corresponding to the same type of tumor markers may have distinct unique expression patterns. By performing blocking operations at different lengths, the most differentiated gene data blocks in each original gene sequence are screened out and regarded as gene expression blocks with unique or special features. The adoption of such unique features in subsequent gene alignment can effectively improve the accuracy of blocked gene comparison.

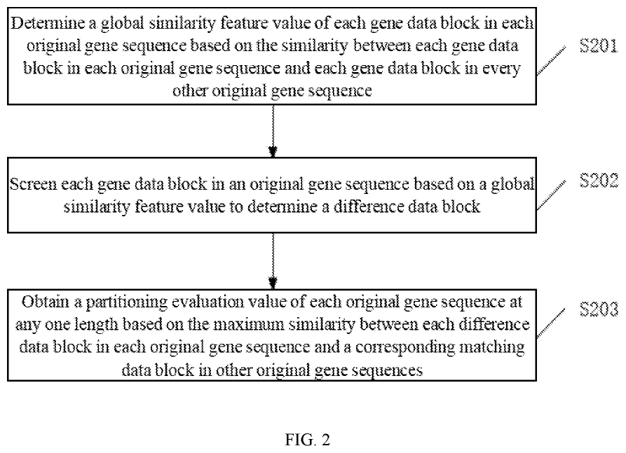

Accordingly, for the blocking results obtained at each different length, the matching similarity and difference between gene data blocks at corresponding positions within the blocking results of the same category are analyzed to evaluate the matching performance, so as to quantify the quality of gene sequence segmentation with different lengths. In this embodiment, as shown in FIG. 2, the acquisition method of segmentation evaluation values is implemented through Step S201 to Step S203.

It should be noted that the performance evaluation of blocking and segmentation is conducted on the segmentation results corresponding to each different length value. Meanwhile, the analysis and identification of gene expression with special or unique features are carried out for the gene information of the same tumor marker. Therefore, in this embodiment, the process of quantitatively analyzing segmentation evaluation values is described based on all original gene sequences corresponding to any one tumor marker, with the segmentation results at an arbitrary length taken as an example.

Step S201, a global similarity feature value of each gene data block in each original gene sequence is determined according to the similarity between each gene data block in each original gene sequence and each gene data block in every other original gene sequences.

For any tumor marker and partitioning results of any length, the gene data blocks of each original gene sequence share the same positional distribution. The matching between gene data blocks at corresponding positions shall also be taken into account during gene feature comparison. Therefore, first, feature analysis is performed on the similarity of gene data blocks in the current segmentation results.

The original gene sequences of any tumor marker are taken as an example under segmentation results of any length, so that any gene data block in an arbitrary original gene sequence is taken as a target data block. The gene data blocks at the corresponding positions in every other original gene sequences are taken as reference data blocks of the target data block. The target data block and the reference data blocks are classified as gene data corresponding to the same type of tumor marker. For example, if the i-th gene data block in the a-th original gene sequence is taken as the target data block, the i-th gene data block in every other original gene sequences under the same segmentation result and for the same tumor marker is defined as the reference data block of the target data block.

Then, the similarity feature factors between the target data block and each reference data block are determined based on the DTW distances therebetween. The calculation method of DTW distance is a well-known technique, which will not be elaborated herein. The DTW distances between the target data block and each reference data block reflect the matching differences between them. The similarity between the target data block and each reference data block can be obtained by performing negative correlation mapping on the calculated DTW distances.

In this embodiment, negative correlation normalization is performed on the DTW distances between the target data block and each reference data block to obtain the similarity feature factors thereof. It should be understood that the method of negative correlation normalization is a well-known technique, and practitioners may select a calculation mode according to specific application scenarios. As a specific example, the similarity feature factor is expressed as

$$L ( {X}_{a , i} , {X}_{b , i} ) = \frac{max ( DTW ) - DTW ( {X}_{a i} , {X}_{b i} )}{max ( DTW ) - min ( DTW )} , {X}_{a , i}$$

represents the i-th gene data block in the a-th original gene sequence, i.e., a target data block; X_{b,i} represents the i-th gene data block in the b-th original gene sequence, i.e., one reference data block of the target data block, and a≈b; L(X_{a,i}, X_{b,i}) represents the similarity feature factor between the target data block and a reference data block, max ( ) represents the maximum value function, min( ) represents minimum value function, max(DTW) represents the maximum value of the DTW distance between the target data block and the reference data block, min(DTW) represents the minimum value of the DTW distance between the target data block and the reference data blockDTW(X_{a,i}, X_{b,i}) represents the DTW distance between the target data block X_{a,i} and the reference data blockX_{b,i}.

A smaller DTW distance value corresponds to a larger value of the similarity feature factor between two gene data blocks, which indicates a higher degree of similarity and matching between the two blocks. The dynamic time warping algorithm is adopted for difference analysis. Optimal matching between two distinct original gene sequences can be realized. This effectively avoids large deviations in calculation results caused by minor changes in a single unit feature, and improves the accuracy of data analysis to a certain extent.

Finally, the global similarity feature value of the target data block is determined according to the number of reference data blocks whose similarity feature factors exceed the preset similarity threshold. The global similarity feature value is a normalized value.

A larger value of the similarity feature factor between the target data block and a reference data block indicates a higher similarity between the two gene data blocks. By counting the number of reference data blocks that share a high similarity with the target data block, the overall similarity feature of the target data block in the database can be reflected. A higher proportion of similar counterparts means the target data block presents general consistency, which further proves that the target data block is not a gene expression block with special or unique features.

Specifically, the number of reference data blocks is counted when the similarity feature factor between the target data block and each reference data block is greater than the preset similarity threshold, which indicates a high gene similarity between them. In this embodiment, the similarity threshold is set to 0.8. Since the similarity feature factor is a normalized value, the similarity threshold ranges from 0 to 1. A value closer to 1 means stricter criteria for evaluating the gene similarity of the target data block, while a value closer to 0 indicates looser evaluation criteria.

In this embodiment, the number of reference data blocks whose corresponding similarity feature factors meet the threshold requirement is counted. The ratio of this number to the total number of all reference data blocks corresponding to the target data block is taken as the global similarity feature value of the target data block, which reflects the proportion of gene data blocks with a high degree of similarity to the target data block.

Step S202, each gene data block in the original gene sequence is screened based on the global similarity feature value to determine a difference data block.

A larger global similarity feature value of the target data block indicates a higher similarity between its genetic information and the genetic information at the corresponding positions in other original gene sequences, meaning the target data block is more likely to represent general gene expression. A smaller global similarity feature value indicates a lower similarity between the above genetic information, and the target data block is more likely to represent gene expression with distinctive feature.

The global similarity feature value of each gene data block in each original gene sequence is obtained using the same method as that for calculating the global similarity feature value of the target data block, and then the gene data blocks are screened according to the global similarity feature values to identify segments corresponding to distinctive gene expression, namely difference data blocks.

Specifically, still taking the target data block as an example, it is defined as a difference data block when its global similarity feature value is less than the preset differential threshold. The differential threshold is set to 0.3, which can be adjusted by the operator according to specific application scenarios. That is, the global similarity feature value of each gene data block is judged separately. A gene data block is regarded as a difference data block if it meets the threshold requirement. A difference data block represents the segment with distinctive or differential gene expression in the corresponding original gene sequence.

Step S203, a partitioning evaluation value of each original gene sequence at any length is obtained according to the maximum similarity between each difference data block in each original gene sequence and correspondingly matching data blocks in other original gene sequences.

By analyzing the similarity between the expressed regions of characteristic genes in an original gene sequence and other original gene sequences, the rationality of the current length-based block partitioning results can be effectively evaluated. It should be noted that for the same tumor marker, each gene data block in every original gene sequence has a corresponding reference data block. Such reference blocks represent the gene data blocks at the identical positions in other original gene sequences, which means each difference data block is also matched with a corresponding reference data block.

Specifically, for any original gene sequence at any length, the partitioning feature factor of each difference data block is determined based on the negative correlation coefficient of the maximum value of the similarity feature factor between each difference data block in the original gene sequence and the corresponding reference data block; and the partitioning evaluation value of any original gene sequence at any length is determined by integrating the partitioning feature factors of all difference data blocks in the original gene sequence.

As a specific example, this embodiment takes the partitioning results corresponding to any length value and any original gene sequence for illustration. For a given length d, the partitioning evaluation value of the a-th original gene sequence can be expressed as

$${W}_{a} ( d ) = {\sum }_{c = 1}^{{N}_{a} ( d )} [ 1 - {L ( {X}_{a , c}^{′} )}_{max} ] ,$$

Wₐ(d) represents the partitioning evaluation value of the a-th original gene sequence under the partitioning result of the length d,X_{a,c}represents the c-th difference data block of the a-th original gene sequence, Nₐ(d) represents the total number of difference data blocks contained in the a-th original gene sequence under the partitioning result of the length d, and

$${L ( {X}_{a , c}^{′} )}_{max}$$

represents the maximum value of the similarity feature factor between the c-th difference data block of the a-th original gene sequence and its corresponding reference data block under the partitioning result with length d.

The negative correlation coefficient of the maximum similarity between each difference data block and its corresponding reference data

$$1 - {L ( {X}_{a , c}^{′} )}_{max}$$

represents the effectiveness of data matching under the current length setting and eliminates the impact of gene data blocks with distinct inherent differences. In subsequent iterations, if most data in one gene data block differ significantly from those in other gene data blocks, this gene data block becomes an outlier. However, the special gene data block may be present in multiple overlapping gene data block intervals. Therefore, the partitioning of the overall gene sequence is adjusted and optimized by using the similarity between gene data blocks as a weight. The partitioning evaluation value of the original gene sequence can characterize the partitioning performance of the original gene sequence at the current length.

Step S300, the optimal length is obtained according to the variation of partitioning evaluation values of all original gene sequences at each length and an adjacent length.

By analyzing the gene similarity expression and distinctive feature expression among different original gene sequences corresponding to the same tumor marker in the partitioning results for each length respectively, the partitioning evaluation value can be used to quantify the partitioning performance evaluation of block processing on different original gene sequences with different lengths. By integrating the partitioning effect evaluation of all original gene sequences under the same length value, the partitioning performance is globally characterized by using the corresponding length. The length with the optimal partitioning effect can be screened out based on the global characterization results.

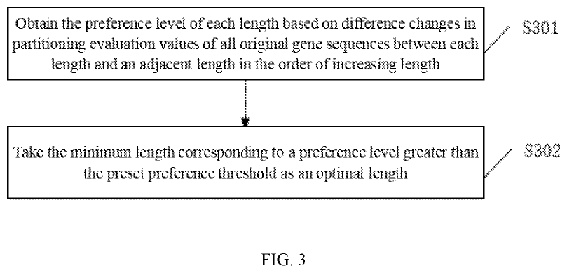

In this embodiment, the acquisition method of the optimal length is as shown in FIG. 3, which can be achieved from Step S301 to Step S302.

Step S301, a preference level of each length is obtained according to the change in differences of partitioning evaluation values of all original gene sequences between each length and an adjacent length in ascending order of length.

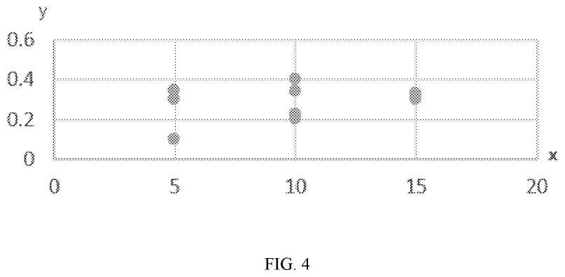

For a given length value, each original gene sequence has a corresponding partitioning evaluation value. FIG. 4 is a coordinate distribution diagram of partitioning evaluation values for partial original gene sequences under partial length values, which is presented by way of example with partial data. In FIG. 4, the horizontal axis x represents the length value, and the vertical axis y represents the partitioning evaluation values of different original gene sequences obtained in the partitioning results at the corresponding length.

In this embodiment, the mean value of the partitioning evaluation values of all original gene sequences is calculated for each length to obtain the partitioning effect characterization value. That is, the partitioning effect characterization value comprehensively reflects the overall partitioning performance achieved with the corresponding length.

In the ascending order of length, the partitioning effect slope value of each length is determined according to the rate of change of the partitioning effect characterization value between the current length and its immediately preceding adjacent length. Each length value corresponds to a partitioning effect characterization value. The method for calculating the partitioning effect slope value of each length is analogous to calculating the slope of data points in a two-dimensional coordinate system. Since the calculation method belongs to the prior art, only a brief description is given herein by way of example.

Specifically, different length values are taken as the horizontal axis and the corresponding partitioning effect characterization values are taken as the vertical axis to construct data points corresponding to each length. Curve fitting is then performed on these data points to obtain the fitted partitioning effect curve. The slope at each data point on the curve is calculated and defined as the partitioning effect slope value of the corresponding length. Methods including data point fitting and slope calculation for points on the curve are well-known in the art and will not be elaborated herein.

for any length, determining a feature evaluation coefficient of the length based on the negative correlation coefficient of partitioning effect slope value difference between the length and the previous adjacent length, and obtaining a preference level of the length according to the feature evaluation coefficient and the partitioning effect characterization value of the length, wherein the feature evaluation coefficient and the partitioning effect characterization value are both positively correlated with the preference level.

By way of example, the preference level corresponding to the t-th lengthQₜcan be expressed as Qₜ=Norm{W̅(t)×exp[−(Kₜ−Kₜ−1)]} wherein Qₜ represents the preference level of the t-th lengthW̅ (t) represents the partitioning effect characterization value of the t-th length, i.e., the mean of partitioning evaluation values of all original gene sequences at the t-th length, Kₜrepresents the partitioning effect slope value of the t-th length, Kₜ₋₁ represents the partitioning effect slope value of the t−1th length, exp[ ] represents the exponential function with the natural constant e as the base, and Norm represents the linear normalization function.

exp[−(Kₜ−Kₜ₋₁)] is the feature evaluation coefficient for the t-th length, reflecting the difference in partitioning effect efficiency values between the t-th length and its immediately preceding length. A smaller value of Kₜ−Kₜ₋₁ indicates that the rate of change in partitioning effect from the (t−1)-th length to the t-th length transitions from rapid to slow, indicating that the improvement in effect at this length value is approaching an inflection point of the length. In this case, the corresponding feature evaluation coefficient becomes larger, and the preference level for the current length increases, leading to a better effect when using this length for gene sequence partitioning.

Meanwhile, the partitioning effect characterization value comprehensively characterizes the partitioning results of all original gene sequences at the current length. A larger value indicates a higher preference level and better performance when adopting this length for gene sequence partitioning. That is, the preference level reflects the accuracy of feature matching when the corresponding length is used to partition different original gene sequences.

It should be noted that when changes in partitioning effect during iterative segmentation are analyzed, it is necessary to analyze the partitioning results under at least multiple different length values. Accordingly, lengths for which the preference level cannot be calculated in this embodiment shall be excluded.

Step S302, a minimum length corresponding to a preference level greater than a preset preference threshold is taken as the optimal length

A larger value of the preference level for different lengths indicates a better performance in feature alignment for gene sequences partitioned with the corresponding length. Therefore, when the preference level exceeds the preset preference threshold, the partitioning effect of the corresponding length is good. Considering that longer block lengths tend to introduce more errors during gene comparison using hash values, the minimum length that meets the threshold requirement is selected as the optimal partitioning length, i.e., the optimal length. In this embodiment, the preference threshold is set to 0.7. Practitioners may adjust this value according to specific application scenarios, and the valid range of the preference threshold is (0,1).

Step S400, the gene sequence to be analyzed is partitioned by using the optimal length, and hash matching is performed between the partitioning results and partitioning results corresponding to tumor markers in the database to obtain the gene sequence screening results of tumor markers in the patient with suspected digestive tract tumors.

Gene sequences are extremely large and complex. Direct full-sequence comparison consumes substantial time and computing resources. Sequence segmentation and hash value generation significantly speed up search and comparison. Hash values generated by the hash function are fixed in length and unique, and can serve as effective indexes for fast lookup and comparison. Segmentation and hash matching enable parallel processing of different sequence segments or data blocks, thus improving the overall processing efficiency and speed. This is particularly important for the processing of large-scale genomic data. In this embodiment, partitioning schemes with different lengths are iterated. Through feature comparison, the partitioning length that best reflects the original gene expressions in the current database is determined adaptively. Feature comparison is then performed on the patient gene information to be matched using this length, so as to achieve more accurate hash matching results.

Specifically, the patient's gene sequence to be analyzed is partitioned into blocks using the optimal length to obtain the gene partitioning results corresponding to the patient, including each data block of the gene sequence to be analyzed. Similarly, each original gene sequence in the database is partitioned into blocks utilizing the optimal length, so as to obtain each gene data block of the original gene sequence, which can be acquired through the aforementioned data analysis procedures.

Further, the hash value of each data block of the gene sequence to be analyzed and the hash value of each gene data block of the original gene sequence can be respectively calculated. Based on these hash values, feature comparison and matching can be performed on each data block of the sequence to be analyzed and the gene data block of each original gene sequence at the same position so as to obtain the matching results. Feature comparison and matching based on hash values is a known technology. Specifically, identical hash values indicate a match, while different hash values indicate a mismatch, which will not be elaborated further herein.

Finally, the screening results of tumor markers for patients with digestive tract disorders can be presented based on the matching results. For example, each type of tumor marker is ranked in descending order by the number of matched gene sequences. This ranking provides significant big data support for relevant physicians.

More preferably, direct hash value matching also involves a large volume of data processing. Therefore, preliminary screening of gene expression blocks with distinctive features can be conducted under the current partitioning results in the database, so as to determine whether to expand the acquired gene sequences for extended matching.

Specifically, for any tumor marker, the difference data block corresponding to the minimum value of the global similarity feature value at the optimal length is taken as the seed data block. This seed data block then characterizes the most distinctive performance among all gene data blocks of the current tumor marker.

Hash matching is performed between the seed data block and each data block derived from partitioning the gene sequence to be analyzed, so as to obtain the matching results between the gene sequence to be analyzed and any tumor marker. Similarly, data blocks with identical hash values are regarded as a match, while data blocks with different hash values are considered a mismatch.

The method for acquiring the matching results is specified as follows: when the proportion of the matching data blocks in the partitioning results of the seed data block and the partitioned gene sequence to be analyzed exceeds the preset proportion threshold, the original gene sequence where the seed data block is located is matched with the gene sequence to be analyzed to obtain the matching result of the tumor marker. The proportion threshold is set to 50%. That is, when the proportion of the matching data blocks exceeds 50%, it indicates that more than half of the distinctive gene expressions are contained in the patient gene information to be compared. To prevent matching errors, extended matching can be performed using longer gene sequences to determine the matching results. When the proportion of the matching data blocks in the partitioning results of the seed data block and the sequence to be analyzed is less than the preset proportion threshold, the data block proportion is directly taken as the matching result.

The concentration proportion of each tumor marker in the gene sequence to be analyzed is acquired based on the matching result of each tumor marker, and the gene sequence screening result of tumor markers is determined according to the descending order of concentration. The concentration ratio can be represented by the proportion of matching data blocks.

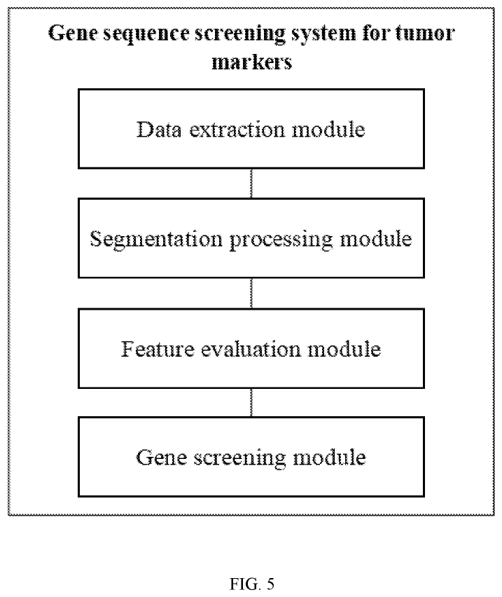

As shown in FIG. 5, the embodiment of the present disclosure further provides a gene sequence screening system for tumor markers, the system comprising:
a data extraction module, configured to acquire a gene sequence to be analyzed from a patient with suspected digestive tract tumors and each original gene sequence of each tumor marker stored in a database, and iteratively partition original gene sequences by using different preset lengths to obtain gene data blocks of each original gene sequence at each length;
a segmentation processing module, configured to obtain a partitioning evaluation value of each original gene sequence at each length according to the matching of data similarity and difference between gene data blocks of each original gene sequence and other original gene sequences for the same type of tumor markers at the same length;
a feature evaluation module, configured to obtain the optimal length according to the change in differences between partitioning evaluation values of all original gene sequences at each length and partitioning evaluation values of all original gene sequences at an adjacent length; and
a gene screening module, configured to partition the gene sequence to be analyzed using the optimal length, and perform hash matching between the partitioning result of the gene sequence to be analyzed and the partitioning result corresponding to the tumor marker in the database, so as to obtain the gene sequence screening results of tumor markers for a patient with suspected digestive tract tumors.

It should be noted that the system provided in the above embodiments is merely illustrated by the partitioning of the foregoing functional modules. In practical applications, the above functions may be allocated to different functional modules as required. That is, the internal structure of the computer device can be partitioned into different functional modules to implement all or part of the functions described above. In addition, the gene sequence screening system for tumor markers provided in the above embodiments shares the same design concept with the gene sequence screening method embodiment of the tumor marker. For its specific implementation process, refer to the method embodiments, which will not be repeated here.

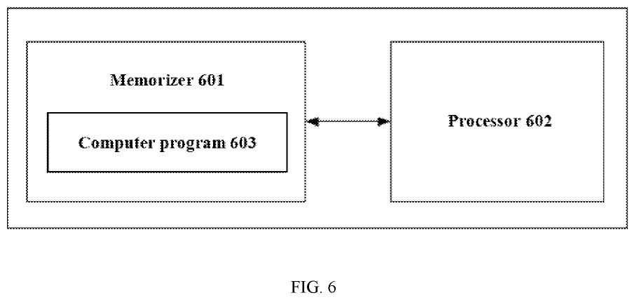

Embodiments of the present application further provide a computer device. Please refer to FIG. 6, which is a structural schematic diagram of a computer device according to an embodiment of the present disclosure. The computer device comprises a memory 601, a processor 602 and a computer program 603 stored in the memory 601 and executable by the processor 602. When the processor 602 runs the computer program 603, the computer device executes any of the aforementioned gene sequence screening methods of the tumor markers.

Embodiments of the present application further provide a computer program product. When the computer program product runs on a computer device, the computer device is enabled to execute any of the aforementioned gene sequence screening methods for tumor markers.

Embodiments of the present application further provide a computer-readable storage medium storing computer program codes. When the computer program codes run on a computer device, the computer device is enabled to execute any of the aforementioned gene sequence screening methods for tumor markers.

It should be understood that the computer device, computer program products and computer-readable storage media provided in the embodiments of the present application are all configured to execute the corresponding methods described above. Therefore, the beneficial effects achieved thereby may refer to those of the aforementioned methods, which will not be elaborated herein.

The above embodiments are merely intended to illustrate the technical solutions of the present application but not limit them. Although the present application has been described in detail with reference to the foregoing embodiments, those skilled in the art shall understand that modifications may still be made to the technical solutions described in the foregoing embodiments, or equivalent replacements may be adopted for part of the technical features. Such modifications or replacements shall not depart from the essence of the technical solutions of the embodiments of the present application and shall all fall within the protection scope of the present application.

What is claimed is:

1. A gene sequence screening method for tumor markers, the method comprising the following steps:
acquiring a gene sequence to be analyzed from a patient with suspected digestive tract tumors and each original gene sequence of each tumor marker stored in a database, and iteratively partitioning the original gene sequences with different preset lengths to obtain gene data blocks of each original gene sequence at each length;
obtaining a partitioning evaluation value of each original gene sequence at each length according to the matching degree of data similarity and difference between gene data blocks of each original gene sequence and gene data blocks of other original gene sequences for the same tumor marker at the same length;
obtaining an optimal length according to the change in differences between the partitioning evaluation values of all original gene sequences at each length and partitioning evaluation values of all original gene sequences at an adjacent length; and
partitioning the gene sequence to be analyzed by using the optimal length, performing hash matching between the partitioning results and partitioning results corresponding to tumor markers in the database to obtain the gene sequence screening results of tumor markers in the patient with suspected digestive tract tumors;
wherein, the step of obtaining a partitioning evaluation value of each original gene sequence at each length according to the matching degree of data similarity and difference between gene data blocks of each original gene sequence and gene data blocks of other original gene sequences for the same tumor marker at the same length specifically comprises:
for any type of tumor marker and any length;
determining a global similarity feature value of each gene data block in each original gene sequence according to the similarity between each gene data block in each original gene sequence and each gene data block in every other original gene sequences;
screening each gene data block in the original gene sequence based on the global similarity feature value to determine a difference data block;
obtaining a partitioning evaluation value of each original gene sequence at any length according to the maximum similarity between each difference data block in each original gene sequence and correspondingly matching data blocks in other original gene sequences;
wherein, the step of obtaining the optimal length according to the change in differences between the partitioning evaluation values of all original gene sequences at each length and the partitioning evaluation values of all original gene sequences at an adjacent length specifically comprises:
obtaining a preference level of each length according to the change in differences of partitioning evaluation values of all original gene sequences between each length and an adjacent length in ascending order of length, wherein a minimum length corresponding to a preference level greater than a preset preference threshold is used as the optimal length;
wherein, the step of obtaining a preference level of each length according to the change in differences of partitioning evaluation values of all original gene sequences between each length and an adjacent length specifically comprises:
calculating a mean value of partitioning evaluation values of all original gene sequences at each length to obtain a partitioning effect characterization value of each length; determining a partitioning effect slope value of each length based on a change rate of a partitioning effect characterization value between each length and a previous adjacent length in ascending order of length; and
for any length, determining a feature evaluation coefficient of the length based on the negative correlation coefficient of partitioning effect slope value difference between the length and the previous adjacent length, and obtaining a preference level of the length according to the feature evaluation coefficient and the partitioning effect characterization value of the length, wherein the feature evaluation coefficient and the partitioning effect characterization value are both positively correlated with the preference level.

2. The gene sequence screening method for tumor markers according to claim 1, wherein the step of determining the global similarity feature value of each gene data block in each original gene sequence according to the similarity between each gene data block in each original gene sequence and each gene data block in every other original gene sequence specifically comprises:
taking any gene data block in any original gene sequence as a target data block, and taking the gene data block at the same corresponding position as the target data block in every other original gene sequence except any of the original gene sequences as a reference data block of the target data block; wherein, the target data block and the reference data block are gene data corresponding to the same type of tumor marker;
determining a similarity feature factor between the target data block and each reference data block based on the dynamic time warping (DTW) distance between the target data block and each reference data block; and
determining the global similarity feature value of the target data block based on the number of corresponding reference data blocks when the similarity feature factor is greater than a preset similarity threshold, wherein the global similarity feature value is a normalized value.

3. The gene sequence screening method for tumor markers according to claim 2, wherein the step of screening each gene data block in the original gene sequence based on the global similarity feature value to determine a difference data block specifically comprises:
determining the target data block as the difference data block when the global similarity feature value of the target data block is less than a preset difference threshold.

4. The gene sequence screening method for tumor markers according to claim 2, wherein the step of obtaining the partitioning evaluation value of each original gene sequence at any length according to the maximum similarity between each difference data block in each original gene sequence and the correspondingly matching data block in other original gene sequences specifically comprises:
determining the partitioning characteristic factor of each difference data block based on the negative correlation coefficient of the maximum value of the similarity feature factor between each difference data block and the corresponding reference data block in the original gene sequence for any original gene sequence at any length; and determining the partitioning evaluation value of any original gene sequence at any length by integrating the partitioning characteristic factors of all difference data blocks in the original gene sequence.

5. The gene sequence screening method for tumor markers according to claim 1, wherein the step of performing hash matching between the gene sequence to be analyzed and the partitioning results corresponding to tumor markers in the database according to the partitioning results to obtain the gene sequence screening result of tumor markers of patients with digestive tract disorders specifically comprises:
for any tumor marker, taking the difference data block corresponding to the minimum value of the global similarity feature value at the optimal length as a seed data block, and performing hash matching based on the seed data block and each data block in the partitioning result of the gene sequence to be analyzed to obtain the matching result between the gene sequence to be analyzed and any of the tumor markers; and
The concentration proportion of each tumor marker in the gene sequence to be analyzed is acquired based on the matching result of each tumor marker, and the gene sequence screening result of tumor markers is determined according to the descending order of concentration.

6. The gene sequence screening method for tumor markers according to claim 5, wherein the matching result between the gene sequence to be analyzed and any of the tumor markers is specifically as follows:
when the proportion of data blocks matching the seed data block in the partitioning result of the gene sequence to be analyzed is greater than the preset proportion threshold, matching the original gene sequence where the seed data block is located with the gene sequence to be analyzed to obtain the matching result of tumor markers.

7. A gene sequence screening system for tumor markers, wherein the system is used for implementing the steps of the gene sequence screening method for tumor markers according to claim 1 and the gene sequence screening system for tumor markers comprises:
a data extraction module, configured to acquire a gene sequence to be analyzed from a patient with suspected digestive tract tumors and each original gene sequence of each tumor marker stored in a database, and iteratively partition original gene sequences by using different preset lengths to obtain gene data blocks of each original gene sequence at each length;
a segmentation processing module, configured to obtain a partitioning evaluation value of each original gene sequence at each length according to the matching of data similarity and difference between gene data blocks of each original gene sequence and other original gene sequences for the same type of tumor markers at the same length;
a feature evaluation module, configured to obtain the optimal length according to the change in differences between partitioning evaluation values of all original gene sequences at each length and partitioning evaluation values of all original gene sequences at an adjacent length; and
a gene screening module, configured to partition the gene sequence to be analyzed using the optimal length, and perform hash matching between the partitioning result of the gene sequence to be analyzed and the partitioning result corresponding to the tumor marker in the database, so as to obtain the gene sequence screening results of tumor markers for a patient with suspected digestive tract tumors.
